(19) United States
(12) Patent Application Publication
(10) Pub. No.: US 2026/0158148 A1
(43) Pub. Date: Jun. 11, 2026

(54) PRODRUG INTENDED FOR THE TREATMENT OF BACTERIAL INFECTIONS RESISTANT TO ANTIBIOTICS OF THE BETA-LACTAM FAMILY

(71) Applicants: Osta Therapeutics, Lyon (FR); Ecole Normale Superieure De Lyon, Lyon Cedex 07 (FR); Université Claude Bernard (UCBL), Villeurbanne (FR); Centre National De La Recherche Scientifique, Paris (FR)

(72) Inventors: Jens Hasserodt, Sainte Foy Les Lyon (FR); Jacques Samarut, Lyon (FR); Mathieu Bordy, Lyon (FR)

(21) Appl. No.: 18/858,853

(22) PCT Filed: Apr. 21, 2023
(86) PCT No.: PCT/EP2023/060405

§ 371 Date: Oct. 22, 2024

(30) Foreign Application Priority Data
Apr. 22, 2022 (FR) FR2203762

(51) Int. Cl. A61K 47/54

(52) U.S. Cl. CPC A61K 47/542

(57) ABSTRACT

A prodrug makes it possible to locally and specifically inhibit a bacterial infection due to a bacterium resistant to antibiotics of the beta-lactam family. This prodrug comprises an antibacterial ingredient from a new therapeutic class, bonded to a bait molecule comprising a functional group that can be cleaved by a beta-lactamase as defined in the classifications of Ambler (1980) and Bush and Jacoby (2010). The antibacterial ingredient is masked, and therefore inactive in the prodrug state, and regains its native structure and antibacterial properties once the prodrug has been cleaved in situ by the pathogenic bacteria.

### CROSS-REFERENCE TO RELATED APPLICATIONS

This application is a national phase entry under 35 U.S.C. § 371 of International Patent Application PCT/EP2023/060405, filed Apr. 21, 2023, designating the United States of America and published as International Patent Publication WO 2023/203187 A1 on Oct. 26, 2023, which claims the benefit under Article 8 of the Patent Cooperation Treaty of French Patent Application Serial No. FR2203762, filed Apr. 22, 2022.

### TECHNICAL FIELD

The present disclosure relates to the field of antibacterial treatments intended to combat antibiotic-resistant infections. The present disclosure relates more particularly to a prodrug that makes it possible to locally and specifically inhibit a bacterial infection due to a bacterium resistant to antibiotics of the beta-lactam family. This prodrug comprises an antibacterial ingredient from a new therapeutic class, bonded to a bait molecule comprising a functional group that can be cleaved by a beta-lactamase as defined in the classifications of Ambler (1980) and Bush and Jacoby (2010). The antibacterial ingredient is derived from trans-cinnamaldehyde; it is masked, and therefore inactive in the prodrug state, and regains its native structure and antibacterial properties once the prodrug has been cleaved in situ by the pathogenic bacteria.

### BACKGROUND

The fight against bacterial infections is a worrying public health issue when it comes to dealing with multi-resistant bacteria. Indeed, many bacteria have developed resistance mechanisms to conventional antibiotics, particularly those in the beta-lactam family. To treat these infections, second-generation antibiotics are administered, but these have side effects. Also, their systemic use (that is, non-local and non-specific) is conducive to the emergence of new resistance against these new molecules.

Strategies for targeting antibacterial molecules at the infection site of antibiotic-resistant bacteria have been proposed, notably by exploiting the resistance mechanism deployed by the bacteria to target the treatment. Such an approach was described by Evans et al. (J. Med. Chem 2019, 62, 4411-4425); it consists of a prodrug that can be activated by the beta-lactamases produced by resistant bacteria. In this way, a conventional antibiotic molecule, ciprofloxacin, is chemically conjugated to a beta-lactam motif recognized by the bacteria and cleaved by the latter so as to release the antibiotic molecule. However, the specificity of this prodrug may be limited by the fact that the molecular structure of the antibiotic agent is in its “active” form within the prodrug. This means that the prodrug is expected to exert antibiotic activity, albeit to a lesser degree, even before it is cleaved by beta-lactamases. That limits the specificity of the prodrug's action on multi-resistant bacteria, but this aspect is essential to avoiding systemic exposure and giving bacteria that are not already multi-resistant the chance to develop resistance mechanisms. While this system provides localized antibacterial action at the site of infection, it does not solve the problem of antibiotic resistance.

There is a real need for new therapeutic strategies to treat infections caused by antibiotic-resistant bacteria. Solutions to address this need must incorporate (1) a new therapeutic target and (2) specific, local action to avoid systemic exposure and the development of resistance.

### BRIEF SUMMARY

The inventors propose a new therapeutic strategy in the fight against antibiotic-resistant bacteria. This approach is based on the antibacterial effect of small molecules targeting a new molecular target, whose mechanism of action is not countered by conventional defense or resistance systems developed by bacteria, such as certain molecules derived from essential oils. However, as these molecules are unstable and their bioavailability low, an innovative delivery system needs to be associated with them, so that the therapeutic solution according to the present disclosure takes the form of a prodrug. This approach consists of a prodrug that enables localized delivery of the active molecule only to the site of interest, the site of infection comprising resistant bacteria, due to an enzymatic reaction specific to multi-resistant bacteria capable of cleaving the prodrug. This prevents any harmful systemic effects of the active ingredient.

Thus, disclosed is a prodrug intended for use in the treatment of bacterial infections resistant to antibiotics of the beta-lactam family comprising an antibacterial ingredient linked to a bait molecule containing a functional group cleavable by a beta-lactamase, wherein it corresponds to formula (I):

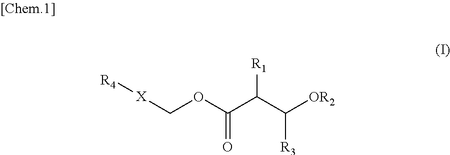

wherein
X is either absent or a spacer group selected from a phenylene, alkenylene or alkynylene group;
R₁ is a group selected from alkyl, alkylsulfonylethyl or alkylaminoethyl, for example, methyl, methylsulfonylethyl or dimethylaminoethyl;
R₂ is a group selected from methyl, a 2,2,2-trifluoroethyl;
R₃ is a masked antibacterial ingredient, the masked active ingredient corresponding to the structural body of trans-cinnamaldehyde devoid of its aldehyde function or one of the variants of this structure having retained antibacterial activity; and
R₄ is a beta-lactam group.

Also described is a pharmaceutical composition comprising a prodrug according to the present disclosure and at least one pharmaceutically acceptable excipient.

Further described is a prodrug according to the present disclosure in the treatment of bacterial infections resistant to antibiotics of the beta-lactam family.

The present disclosure differs from existing solutions by the choice of antibacterial active ingredient and by the fact that this active ingredient is masked within the prodrug (and therefore rendered inactive in prodrug form). In fact, the inventors have developed an approach to exploit the antibacterial properties of small molecules whose instability and low bioavailability have hitherto been an obstacle to their therapeutic use, as is the case for those derived from essential oils (EOs). The latter are known for their antibacterial activity and their novel mechanism of action, which is unlikely to induce resistance in bacteria. Thus, the aim of the present disclosure is to make available a new class of antibacterial (pro-) drugs that are effective and safe (not inducing new resistance) for treating infections caused by beta-lactam-resistant bacteria.

The drug in question is in the form of a prodrug comprising the antibacterial ingredient derived from a small molecule carrying an aldehyde function; this function is exposed to multiple mechanisms of rapid modification by the patient's metabolism, resulting in poor bioavailability of the small molecules. To guarantee their arrival at the site of infection, they need to be incorporated into molecular constructs of suitable stability, water solubility, and bioavailability. The active ingredient is thus modified by conjugation with a bait molecule. Another advantage of this system is that it masks the activity of the active ingredient, but ensures its release in the presence of bacteria resistant to antibiotics of the beta-lactam family. Once released, the molecule regains its active form and antibacterial properties. Active ingredient delivery is therefore targeted to the site of infection, avoiding the side effects and risks of resistance development associated with systemic distribution of the antibacterial agent.

Release of the active ingredient depends on the action of the resistant bacteria. In fact, the prodrug includes a group recognized by beta-lactamases secreted by the resistant pathogenic bacteria, which catalyze the cleavage of the prodrug's bait molecular unit, the beta-lactam unit. The collapse of that unit releases the antibacterial ingredient and delivers an active molecule to the site of infection. The prodrug thus acts as a Trojan horse, as the bacterium, by activating its defense system—the secretion of a beta-lactamase-type enzyme—induces the release of the antibacterial molecule that will destroy and/or inhibit it.

In more detail, between the beta group and the active ingredient (R3 group), the prodrug comprises an N/O-acetal-oxycarbonyl group linked to an X spacer group. It is this N/O-acetal-oxycarbonyl group that ensures the incorporation and masking of the active ingredient, as well as its release following the collapse of the prodrug caused by the action of the target enzyme. This is an original feature of the prodrug structure, on which the innovative properties of the invention are based in relation to similar prodrug molecules in the prior art: release is efficient but the prodrug is stable to spontaneous hydrolytic degradation in the absence of the target enzyme. This property also ensures that the prodrug cannot exert any intrinsic antibiotic action, since the structure of the active ingredient is not recovered in the absence of cleavage. These two properties make it possible to offer the clinical use of prodrugs according to the present disclosure.

The system according to the present disclosure therefore relies on the local, immediate and specific action of an antibacterial molecule from a new therapeutic class that is not likely to induce new resistance. An example of such a molecule is trans-cinnamaldehyde, the active ingredient in cinnamon essential oil.

### BRIEF DESCRIPTION OF THE DRAWINGS

FIG. 1 shows a representation of the OSTA-1 prodrug synthesis method.

FIG. 2 shows the UV absorbance spectrum at 285 nm of the OSTA-1 molecule in vitro.

FIG. 3 shows the mechanism of the active ingredient activation after the collapse of the OSTA-1 prodrug.

### DETAILED DESCRIPTION

A first object of the present disclosure concerns a prodrug intended for use in the treatment of bacterial infections resistant to antibiotics of the beta-lactam family comprising an antibacterial active ingredient linked to a bait molecule comprising a functional group cleavable by a beta-lactamase, wherein it corresponds to formula (I):

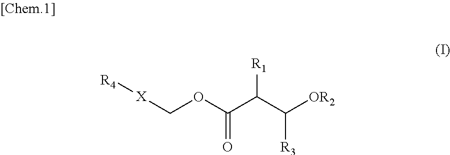

wherein:
X is either absent or a spacer group selected from a phenylene, alkenylene or alkynylene group;
R₁ is a group selected from alkyl, alkylsulfonylethyl or alkylaminoethyl, for example, methyl, methylsulfonylethyl or dimethylaminoethyl;
R₂ is a group selected from methyl, a 2,2,2-trifluoroethyl;
R₃ is a masked antibacterial ingredient, the masked active ingredient corresponding to the structural body of trans-cinnamaldehyde devoid of its aldehyde function or one of the variants of this structure having retained antibacterial activity; and
R₄ is a beta-lactam group.

The term “prodrug” refers to a drug precursor. This is an inactive/low-activity form of a molecule that is metabolized in vivo into an active metabolite. In the present disclosure, the prodrug consists of a masked antibacterial ingredient and a bait molecule; the antibacterial ingredient is therefore the metabolite.

The active ingredient is a small molecule drug with antibacterial properties. It is masked in the prodrug, that is, in an inactive form. Such an active ingredient may be derived from various types of plant, including plants or lichens, and may, in particular, be derived from an essential oil, or from animals, in particular, marine animals.

In a preferred embodiment of the present disclosure, the antibacterial active ingredient is derived from an EO with known antibacterial properties, trans-cinnamaldehyde as shown below, or one of its variants. By “trans-cinnamaldehyde variant” is meant a molecule that has the same structural body as cinnamaldehyde, exhibits similar, identical or superior antibacterial activity and/or confers enhanced stability on the prodrug. Such variants can, in particular, be substituted at the phenyl unit, for example, by replacing an H with a methoxy, ethoxy, alkylamino group such as dimethylamino, or an acylamino group.

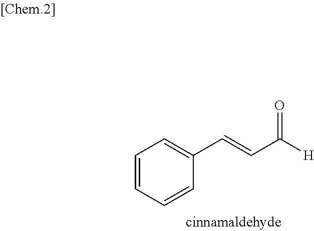

Within the prodrug, the active ingredient is rendered inactive by masking its aldehyde function. This modification has two functions: it enables binding to the bait molecule, and it masks (temporarily suppresses) the antibacterial activity of the active ingredient while in prodrug form.

In the context of the present disclosure, masking is achieved by transforming the aldehyde function into an N/O acetal whose nitrogen atom is acylated (more precisely, “oxy-carbonylated”) to give the acetal greater stability against spontaneous hydrolysis (degradation), and to enable the release of this oxy-carbonylated N/O acetal as a carbamate (good leaving group) upon activation of the bait portion of the prodrug by a target beta-lactamase. “R₃” then represents a phenyl-vinyl unit.

A “bait molecule” in the sense of the present disclosure means a group recognized by bacteria and capable of inducing activation of the bacteria's defense functions against antibiotics of the beta-lactam family. These defense functions consist in secreting an enzyme capable of cleaving the beta-lactam nucleus present in this family of antibiotics.

The bait molecule according to the present disclosure comprises an R₄ group recognized by bacteria resistant to beta-lactams. This R₄ group is a beta-lactam group that mimics the presence of an antibiotic. As explained above, recognition of this group acts as a signal triggering the secretion of a beta-lactamase by the bacterium, which acts in defense. The bait molecule also comprises the central group of formula (I) (N/O-acetal-oxycarbonyl group) and optionally a spacer group X.

A particularly innovative aspect of the present disclosure lies in the structure of the cleavable functional group-which corresponds to the central N/O-acetal-oxycarbonyl group of formula (I)—which fulfills three functions: (i) binding of the active ingredient to a beta-lactam group, (ii) masking of the active ingredient's antibacterial activity by its incorporation into the N/O-acetal-oxycarbonyl unit, and (iii) activation of antibacterial activity by cleavage of the beta-lactam group after secretion of a beta-lactamase by the bacteria, followed by self-collapse of the structure, leading to release of the active ingredient in a restored active form with new-class antibiotic properties. In fact, it is remarkable that the cleavable group has the property of allowing the active ingredient that has been modified to synthesize the prodrug to regain its native form and antibacterial properties after being released. Here, the active ingredient carries an aldehyde function which is linked to the bait molecule at R₃, as shown in formula (I). The aldehyde function of the active ingredient is reformed in situ. This mechanism of action is shown in FIG. 3.

In a preferred embodiment of the present disclosure, the beta-lactam group (R₄ group) is derived from a cephalosporin, penicillin or carbapenem. This group is cleavable by a beta-lactamase, which may be a carbapenemase.

There are many R₄ groups described in the literature, and the person skilled in the art will be able to identify them. A particular example is cefalotin.

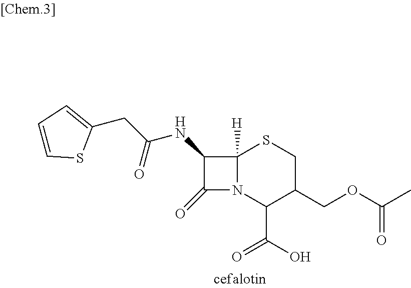 [SMILES: CC(=O)OCC1CS[C@@H]2[C@H](NC(=O)Cc3cccs3)C(=O)N2C1C(=O)O]

The R₄ group may be more generally represented by the following formula (II):

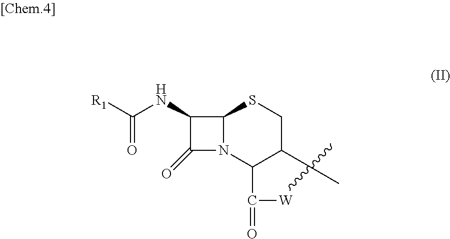

wherein R₇ is selected from alkyl, benzyl, substituted or unsubstituted aryl groups.

Examples of particular R₇ groups are shown below:

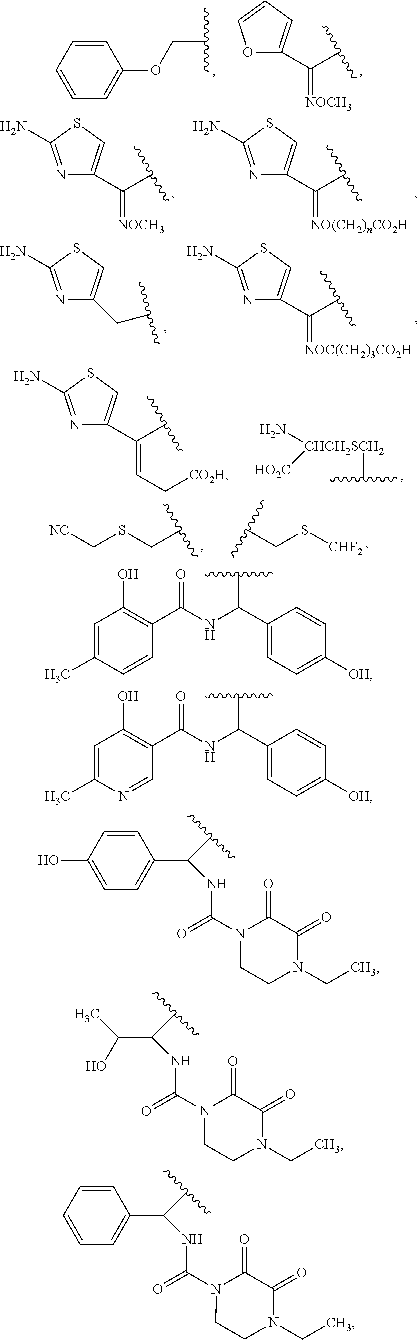

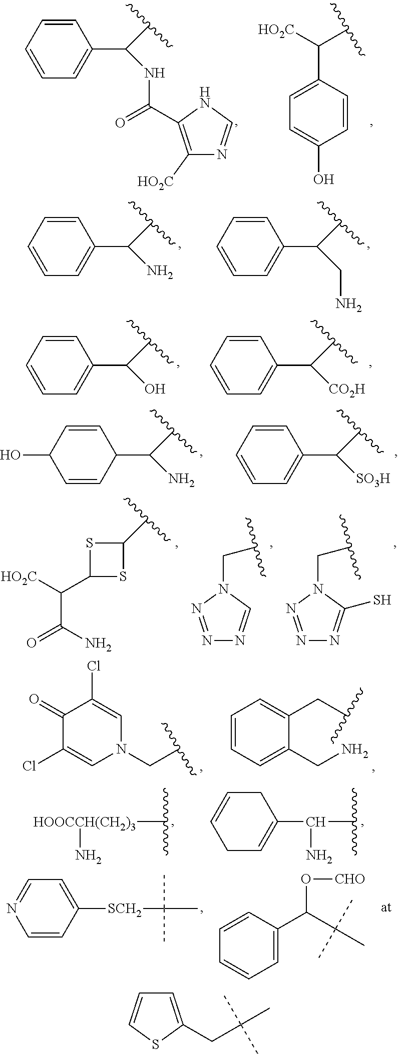

The spacer group X modulates the prodrug's properties according to the choice of group X and the active ingredient associated with the prodrug. Its presence is optional. In the configuration where the X group is absent, the R₄ group is directly linked to the N/O-acetal-oxycarbonyl group.

Examples of prodrugs according to the present disclosure are the OSTA-1, OSTA1b, OSTA-5, OSTA-6 and OSTA-7 molecules shown below:

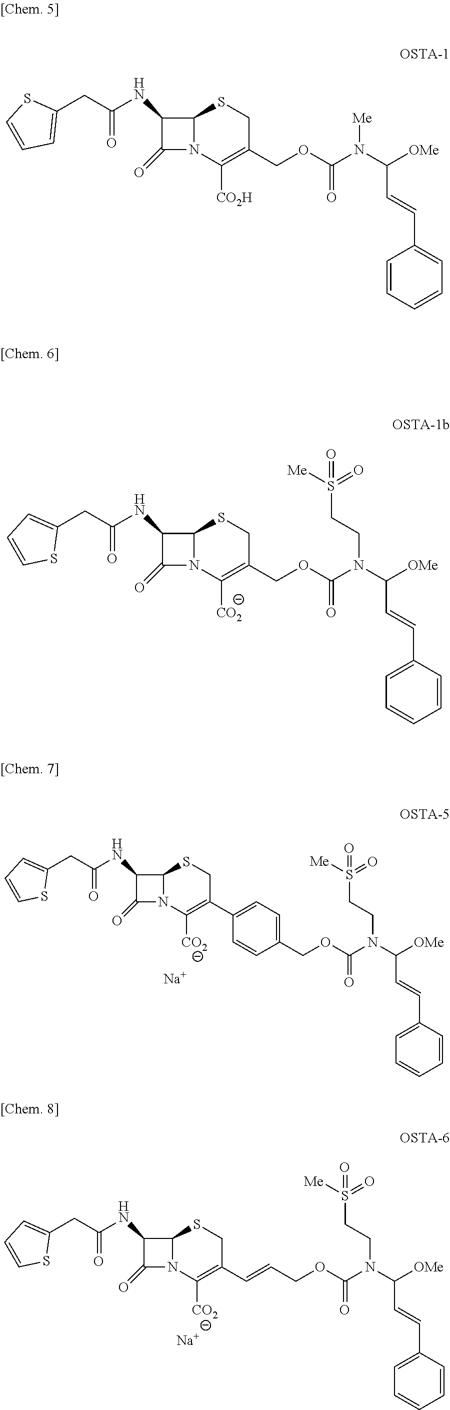

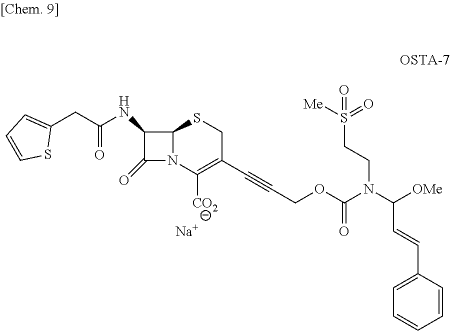

A second object of the present disclosure relates to a pharmaceutical composition comprising a prodrug as defined above and at least one acceptable excipient.

A third object of the present disclosure concerns the use of a prodrug as previously defined in the treatment of bacterial infections resistant to antibiotics of the beta-lactam family.

Bacterial infections resistant to beta-lactam antibiotics are mainly caused by Gram-negative bacteria capable of producing beta-lactamases. But some Gram-positive bacteria, such as MRSA, can also produce beta-lactamases.

The prodrugs according to the present disclosure are particularly aimed at:
gram-negative bacteria belonging to one of the following families: Moraxellaceae, Legionellaceae, Enterobacteriaceae, Pseudomonadaceae, Alcaligenaceae, Sphingomonadaceae, Pasteurellaceae, Neisseriaceae, Pasteurellaceae, Flavobacteriaceae, Bacteroidaceae, Fusobacteriaceae, Burkholderia cepacea, Cronobacter malonaticus, Cronobacter sakazakii, Helicobacter pylori, Micrococcus luteus, Prevotella, Proteus mirabilis, Proteus vulgaris G, Salmonella enteritidis G-, Salmonella enterica serovar typhi, Salmonella typhimuriu, Salmonella enterica serovar paratyphi A, Vibrio fluvialis (MDR), Vibrio parahaemolyticus, Acinetobacter baumannii;
gram-negative bacteria belonging to one of the following genera: Klebsiella, Serratia, Citrobacter, Salmonella, Shigella, Haemophilus, Campylobacter, Legionella, Sphingomonas, Moraxella, Kingella, Pasteurella, Capnocytophaga, Neisseria, Bacteroides, Enterobacter, Pseudomonas, Fusobacterium, Achromobacter, Escherichia and Acinetobacter;
gram-negative bacteria belonging to one of the following species: Klebsiella pneumoniae, Klebsiella oxytoca, Acinetobacter baumannii, Acinetobacter calcoaceticus, Serratia marcescens, Citrobacter freundii, Citrobacter koseri, Haemophilus influenzae, Haemophilus parainfluenza, Campylobacter jejuni, Campylobacter coli, Legionella pneumophila, Legionella longbeachae, Legionella bozmanii, Legionella micdadei, Enterobacter cloacae, Escherichia coli, Escherichia hermannii, Pseudomonas aeruginosa, Pseudomonas fluorescens, Pseudomonas putida, Pseudomonas stutzeri, Achromobacter xylosoxidans, Achromobacter denitrificans, Sphingomonas paucimobilis, Moraxella catarrhalis, Kingella kingae, Pasteurella multicida, Capnocytophaga canimorsus, Neisseria gonorrhoeae, Neisseria actamica and Bacteroides fragilis; and
in particular, Pseudomonas aeruginosa and Acinetobacter baumannii (especially Acinetobacter baumannii 5377 or mucoid Acinetobacter baumannii).

The following gram-positive bacteria:
Families: Staphylococcaceae, Micrococcaceae, Streptococcaceae, Enterococcaceae, Bacillaceae, Clostridiaceae, Listeriaceae, Corynebacteriaceae;
Genera: Staphylococcus, Micrococcus, Streptococcus, Enterococcus, Bacillus, Clostridium, Listeria, Corynebacterium; and
Species (examples): Staphylococcus aureus, Staphylococcus epidermitis, Staphylococcus saprophyticus, Micrococcus luteus, Streptococcus pyogenes, Streptococcus agalactiae, Streptococcus pneumoniae, Streptococcus viridans, Enterococcus faecalis, Enterococcus faecium, Bacillus anthracis, Clostridium difficile, Listeria monocytogenes, Corynebacterium diphteriae.

Other non-Gram-colorable bacteria:
Family: Mycobacteriaceae;
Genus: Mycobacterium; and
Species: Mycobacterium tuberculosis, Mycobacterium leprae,
Mycobacterium abscessus, Mycobacterium ulcerans.

These infections particularly affect the digestive tract, urinary tract, pulmonary tract, or skin and mucous membranes, and may involve a particular organ or be generalized. Prodrugs according to the present disclosure can be used to treat all these forms of infection.

Examples of infections (with or without comorbidity such as genetic factors, immunodepression, etc.) for which the molecules according to the present disclosure are particularly attractive are respiratory tract infections (with or without comorbidity): Pneumonia, HAP, VAP, tuberculosis, sinusitis, cystic fibrosis; Blood or blood-borne infections: Bacteremia, sepsis; urinary tract infections: Cystitis, pyelonephritis; skin and soft tissue infections: Abscesses, boils, impetigo, non-healing wounds, necrotizing infections; surgical site infections; infections associated with prostheses, medical devices (catheters, tubes, etc.); gastrointestinal infections; sexually-transmitted infections; infections of the central or peripheral nervous system, heart, eyes, ears, mouth, bones and joints.

The present disclosure will be better understood upon reading the examples which follow, provided by way of illustration, and in no way considered to be limiting on the scope of the present disclosure.

### EXAMPLES

### Example 1: Synthesis of Compound a (OSTA-1)

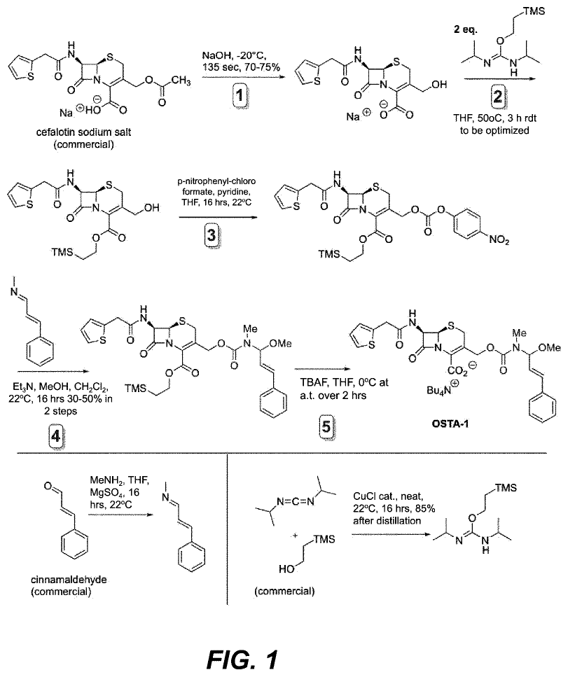

The synthesis protocol for the OST1 prodrug molecule comprises five steps and two auxiliary steps. It is shown in FIG. 1.

### Example 2: Functional Analysis of the OSTA-1 Molecule

### A—In-Vitro Cleavage of the OSTA-1 Molecule

Cleavage of the OSTA-1 prodrug was tested in vitro by contact with a beta-lactamase in saline aqueous media.

### Experimental Conditions:

The cleavage test is performed in physiological saline buffer (Phosphate Buffer Saline, PBS) at pH 7.0. The OSTA-1 molecule is added at a concentration of 100 microM, along with commercial beta-lactamase at a concentration of 1 unit per mL. Incubation is carried out at 37° C. The appearance of free trans-cinnamaldehyde in the medium is then monitored by UV absorption at 285 nm.

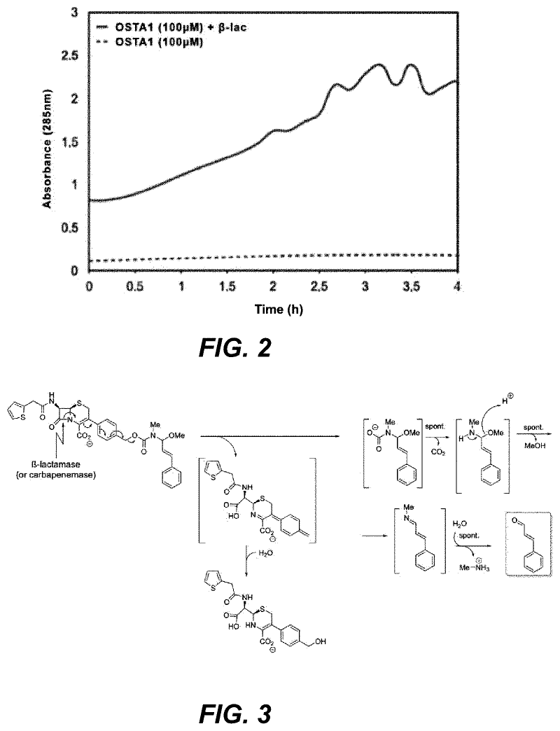

The results are shown in FIG. 2.

The release of the active ingredient, in this case trans-cinnamaldehyde, was monitored by UV absorption at 285 nm. The progress of the release of the prodrug molecule's active ingredient (100 microM) was observed, in the presence and absence of beta-lactamase. It is clear that the active ingredient is released in significant quantities after two hours of reaction in the presence of beta-lactamase. In the absence of beta-lactamase, no spontaneous release of the active ingredient is observed.

Conclusion: The OSTA-1 prodrug molecule functions as expected: It is cleavable by beta-lactamase and releases the active ingredient.

B—Activation of the Active Ingredient after Prodrug Collapse

The mode of activation of the active ingredient is shown in FIG. 3, which shows the mechanism of collapse of the OSTA-1 prodrug and the release of cinnamaldehyde in its active native form.

1. A prodrug useful in treating bacterial infections resistant to antibiotics of the beta-lactam family, the prodrug comprising:
an antibacterial active ingredient linked to a bait molecule containing a functional group cleavable by a beta-lactamase, the prodrug corresponding to formula (I): 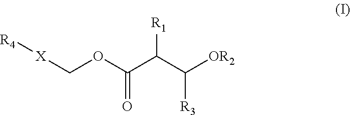
wherein
X is either absent or a spacer group selected from a phenylene, alkenylene or alkynylene group;
R₁ is a group selected from alkyl, alkylsulfonylethyl or alkylaminoethyl, for example, methyl, dimethyl, methylsulfonylethyl or dimethylaminoethyl;
R₂ is a group selected from methyl, a 2,2,2-trifluoroethyl;
R₃ is a masked antibacterial ingredient, the masked antibacterial ingredient corresponding to trans-cinnamaldehyde's structure devoid of its aldehyde function or a variant of said structure that has retained antibacterial activity; and
R₄ is a beta-lactam group derived from a cephalosporin, penicillin or carbapenem.

2. The prodrug of claim 1, wherein the R₄ is a cephalosporin, penicillin, or carbapenem derivative.

3. The prodrug of claim 2, wherein X is absent.

4. The prodrug of claim 3, wherein the prodrug is selected from the molecules:

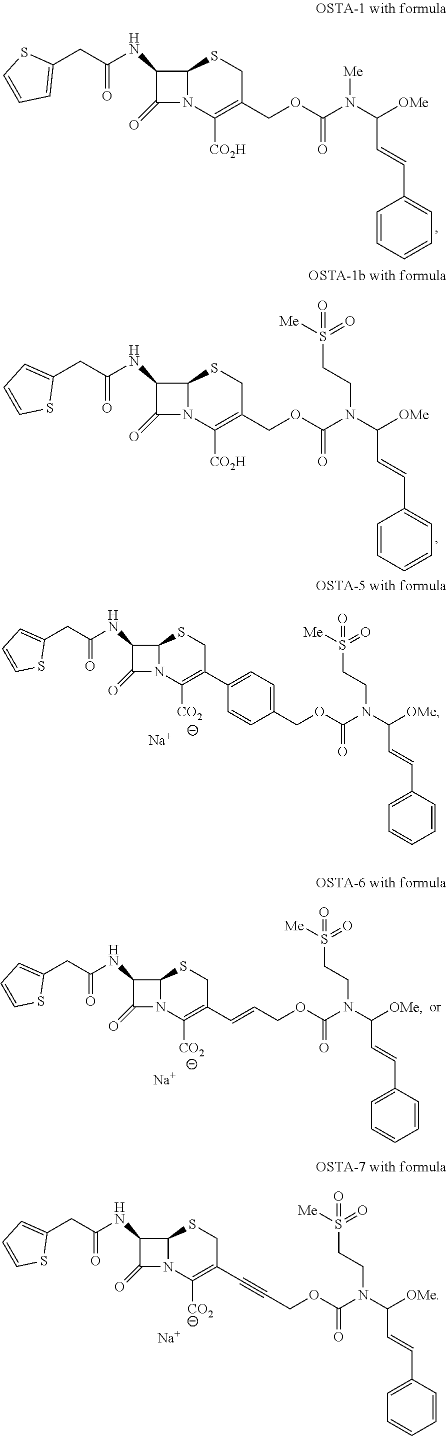

5. A pharmaceutical composition comprising a prodrug according to claim 4 and at least one acceptable excipient.

6. (canceled)

7. The prodrug of claim 1, wherein R₁ is a group selected from methyl, dimethyl, methylsulfonylethyl, or dimethylaminoethyl.

8. The prodrug of claim 1, wherein X is absent.

9. The prodrug of claim 1, wherein the prodrug is selected from:

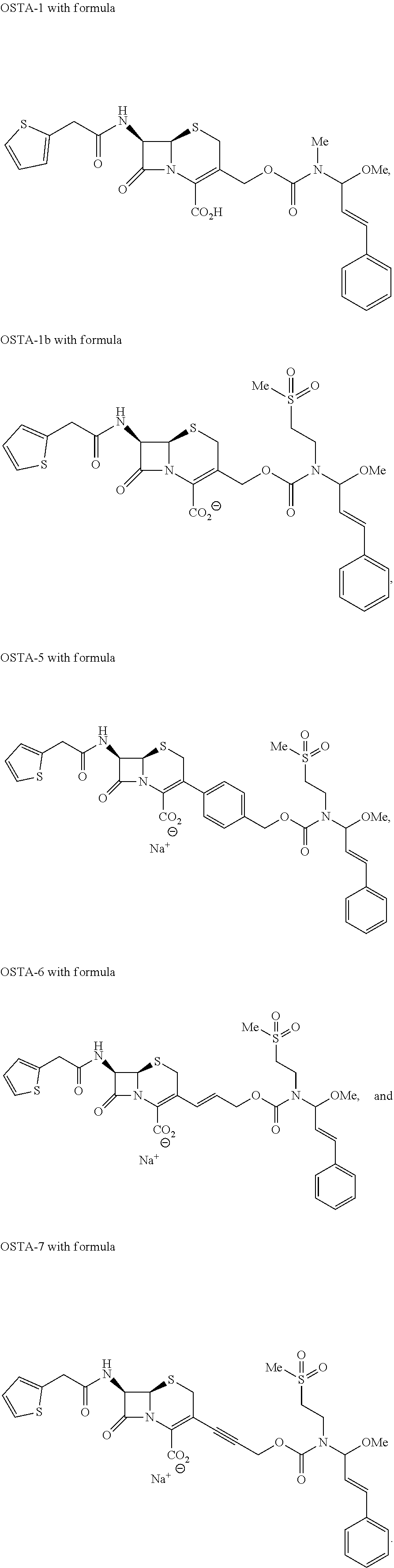

10. A pharmaceutical composition comprising the prodrug of claim 1 and at least one acceptable excipient.

11. A method of treating a subject having a bacterial infection resistant to beta-lactam antibiotics, the method comprising:
administering the prodrug of claim 1 to the subject so as to treat the bacterial infection.

12. A method of treating a subject having a bacterial infection resistant to beta-lactam antibiotics, the method comprising:
administering the prodrug of claim 2 to the subject so as to treat the bacterial infection.

13. A method of treating a subject having a bacterial infection resistant to beta-lactam antibiotics, the method comprising:
administering the prodrug of claim 3 to the subject so as to treat the bacterial infection.

14. A method of treating a subject having a bacterial infection resistant to beta-lactam antibiotics, the method comprising:
administering the prodrug of claim 4 to the subject so as to treat the bacterial infection.

15. A method of treating a subject having a bacterial infection resistant to beta-lactam antibiotics, the method comprising:
administering the prodrug of claim 7 to the subject so as to treat the bacterial infection.

16. A method of treating a subject having a bacterial infection resistant to beta-lactam antibiotics, the method comprising:
administering the prodrug of claim 8 to the subject so as to treat the bacterial infection.

17. A method of treating a subject having a bacterial infection resistant to beta-lactam antibiotics, the method comprising:
administering the prodrug of claim 9 to the subject so as to treat the bacterial infection.
